(19) United States
(12) Patent Application Publication
(10) Pub. No.: US 2018/0096483 A1
(43) Pub. Date: Apr. 5, 2018

(54) METHOD OF PRESENTING HEALTH CARE INFORMATION

(71) Applicant: MyDoc Pte. Ltd., Singapore (SG)

(72) Inventors: Snehal Patel, Singapore (SG); Vas Metupalle, Singapore (SG)

(73) Assignee: MyDoc Pte. Ltd., Singapore (SG)

(21) Appl. No.: 15/820,551

(22) Filed: Nov. 22, 2017

Related U.S. Application Data
(62) Division of application No. 14/293,398, filed on Jun. 2, 2014.
(60) Provisional application No. 61/830,240, filed on Jun. 3, 2013.

(51) Int. Cl. G06T 7/20; H04N 19/00; G06F 19/00

(52) U.S. Cl. CPC G06T 7/20; G06F 19/00; A61B 34/10; H04N 19/00

(57) ABSTRACT

A method of presenting health care information comprises creating patient health data for a patient, storing the patient health data on a server, accessing the patient health data by one of the patient and a health care provider on a corresponding computer, wherein the corresponding computer is connected to the server over the internet, and presenting the patient health data accessed in sections searchable by icons presented on the corresponding computer. The patient health data has transcribable portions, and graphical data may be incorporated into the presentation in graphical form.

### RELATED APPLICATIONS

This patent application is a divisional patent application of U.S. patent application Ser. No. 14/293,398 filed on Jun. 2, 2014, which claims priority benefit of U.S. Provisional Patent Application No. 61/830,240, filed on Jun. 3, 2013, the disclosures of which are expressly incorporated herein in their entireties.

### FIELD OF THE INVENTION

This invention relates to methods and systems of presenting health care information, and more particularly to methods and systems for improving doctor-patient relationships and in the evaluation of patient health data.

### BACKGROUND OF THE INVENTION

The doctor-patient relationship is central to the practice of healthcare and is essential for the delivery of high-quality health care in the diagnosis and treatment of disease. Universities teach students from the beginning to maintain a professional rapport with patients, uphold patients' dignity, and respect their privacy. A patient must have confidence in the competence of their physician and must feel that they can confide in him.

The quality of the patient-physician relationship is important to both parties. The better the relationship in terms of mutual respect, knowledge, trust, shared values and perspectives about disease and life, and time available, the better will be the amount and quality of information about the patient's disease transferred in both directions, enhancing accuracy of diagnosis and increasing the patient's knowledge about the disease. Where such a relationship is poor the physician's ability to make a full assessment is compromised and the patient is more likely to distrust the diagnosis and proposed treatment, causing decreased compliance by the patient in following the medical advice.

A dilemma may arise in situations where determining the most efficient treatment, or resistance to undergoing treatment creates a disagreement between the physician and the patient, for any number of reasons. There may also be differences in opinion between the doctor and patient in how formal or casual the doctor-patient relationship should be. Also, since physicians see many patients, some they have not seen in weeks, months or even years, it can be difficult if not impossible for a physician to remember all relevant information about each patient. Patients are often referred from one physician to another, as in the case of a general practitioner referring to a patient to a specialist, or in the case of complex cases where a second opinion is sought. The patient has to repeat a description of his symptoms to the second physician, which can be tedious and frustrating for the patient. Moreover, this repetition of medical history increases the chances of error.

Since the patient-doctor relationship is important, it would be desirable to enhance that relationship by helping to increase the likelihood that the doctor and the patient have a good interpersonal relationship. Further, as noted above, there is inefficiency in the way information about a patient is currently presented to physicians. It would therefore be desirable to provide a method of enhancing information presentation, especially to the physician, which takes advantage of modern computer technology so that a quicker and more accurate diagnosis may be reached, and more generally waste and delay in presentation of information to health care providers is reduced.

### SUMMARY OF THE INVENTION

In accordance with a first aspect, a method of presenting health care information comprises creating patient health data for a patient, storing the patient health data on a server, accessing the patient health data by one of the patient and a health care provider on a corresponding computer, wherein the corresponding computer is connected to the server over the Internet, and presenting the patient health data accessed in sections searchable by icons presented on the corresponding computer. The patient health data has transcribable portions, and graphical data may be incorporated into the presentation in graphical form. In accordance with another aspect, a psychometric score indicative of compatibility between the patient and the health care provider is based on a comparison of a patient profile and a health care provider profile, and information about the psychometric score is presented to the patient.

From the foregoing disclosure and the following more detailed description of various embodiments it will be apparent to those skilled in the art that the present invention provides a significant advance in the technology of health care information presentation and managing doctor-patient relations. Particularly significant in this regard is the potential the invention affords for providing a method for presenting health care information which enhances the doctor-patient relationship. Additional features and advantages of various embodiments will be better understood in view of the detailed description provided below.

### BRIEF DESCRIPTION OF THE DRAWINGS

FIG. 1 is overview of the module for enhancing doctor-patient relationships in accordance with one embodiment, showing patient and health care providers creating patient health data (PHD).

FIG. 2 is a schematic flowchart showing patient inputs.

FIG. 3 is a schematic flowchart showing health care provider (HCP) inputs.

FIG. 4 is an example of one embodiment of PHD organized by icons and displayed on a mobile device.

FIG. 5 is a schematic flow chart representing a module for enhancing the doctor-patient relationship by use of a pre-screening process based on psychometric profiles in accordance with another preferred embodiment.

FIG. 6 is a schematic high level back end view of the module of FIG. 1, based on a platform of a cloud server and mobile devices.

FIG. 7 is a schematic of the client portion of the back end view of FIG. 6.

FIG. 8 is a schematic of the cloud services portion of the back end view of FIG. 6.

FIG. 9 is a schematic of the representational state transfer application programming interfaces (REST API).

FIG. 10 is a schematic of a link to an administrative application.

FIG. 11 is a representative display of administrative information in summary form pushed out by the administrative application of FIG. 10.

FIG. 12 is an example of a detailed description of the administrative information of FIG. 11.

It should be understood that the appended drawings are not necessarily to scale, presenting a somewhat simplified representation of various features illustrative of the basic principles of the invention. The specific design features of the mobile device as disclosed here, including, for example, the specific icons provided, will be determined in part by the particular intended application and use environment. Certain features of the illustrated embodiments have been enlarged or distorted relative to others to help provide a clearer understanding. In particular, thin features may be thickened, for example, for clarity of illustration. All references to direction and position, unless otherwise indicated, refer to the orientation illustrated in the drawings.

### DETAILED DESCRIPTION OF CERTAIN EMBODIMENTS

It will be apparent to those skilled in the art, that is, to those who have knowledge or experience in this area of technology, that many uses and design variations are possible for the method for presenting health care information disclosed here. The following detailed discussion of various alternate features and embodiments will illustrate the general principles of the invention with reference to a method for presenting health care information suitable for use by both general practitioners and specialists, as well as other health care providers. Other embodiments suitable for other applications will be apparent to those skilled in the art given the benefit of this disclosure.

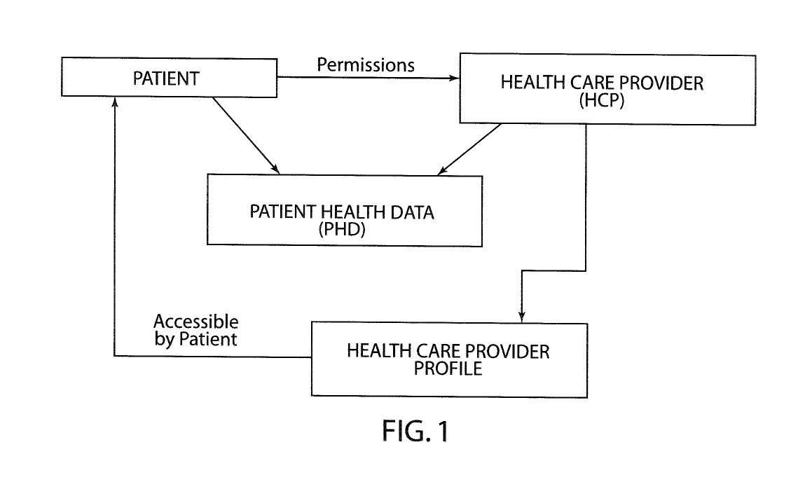

The module disclosed herein aims to identify and organize all medical records and interactions between patients and healthcare professionals into sensible patient led medical records that can be shared efficiently. Each patient and each health care practitioner accesses the patient health data with their own corresponding computer. Preferably the module takes advantage of a remote server for data storage and separate computers with Internet connections to the server, preferably with the option of a wireless connection so that the device used can be lightweight and handheld. This is particularly desirable for the computer used by the health care provider so as to form a convenient mobile clinical platform. Turning now to the drawings, FIG. 1 shows an overview of the module for enhancing doctor-patient relationships in accordance with one embodiment, showing patient and health care providers (HCPs) creating patient health data (PHD). Health care providers, as that term is used herein, is understood in a broad sense to mean not just doctors but also any person or institution who helps in identifying or preventing or treating illness or disability, such as, for example, nurses, hospitals, medical technicians, rehabilitation coaches, caregivers and other health care professionals. Patients log into the module to provide information about their unique PHD. Typically such patient health care data comprises quantifiable (objective) data and non-quantifiable (principally subjective) data. Examples comprise audio messages, live chat, audio-video streaming systems, electronic medical records, hospital information system data, medical images, lab information, scheduling data, and psychometric profiles. Health care providers can also input both quantifiable and non-quantifiable data to the patient health data, optionally upon receipt of permission from the patient.

Health care providers (HCP) also complete a health care provider profile. The HCP profile typically comprises, for example, at least one of: a geographic location; a healthcare plan accepted by the HCP; a healthcare provider organization; a time indicator that indicates when a doctor is available; a new-patient indicator indicating if a doctor is accepting new patients; a satisfaction rating; an amenities rating; a transportation rating; a dispute rating; a procedure cost; and an experience rating. HCP profiles can serve as a form of advertisement for the HCPs, and could be visible to the patient and other prospective patients.

A large quantity of medical records are generated for each patient, and these records are sometimes in a readily quantifiable form and sometimes they are not. Also, data comes from disparate networks with different formats. The module disclosed herein advantageously integrates multiple applications including live chat, audio-video streaming systems, especially voice messages, electronic medical records (EMR), hospital information system data (HIS), medical imaging system data, lab information system data and patient monitoring device data onto a single mobile user interface. The platform enables interoperable patient data, which can be stored on cloud storage systems and viewed on any computer, most preferably a mobile device, giving flexibility in point of care delivery.

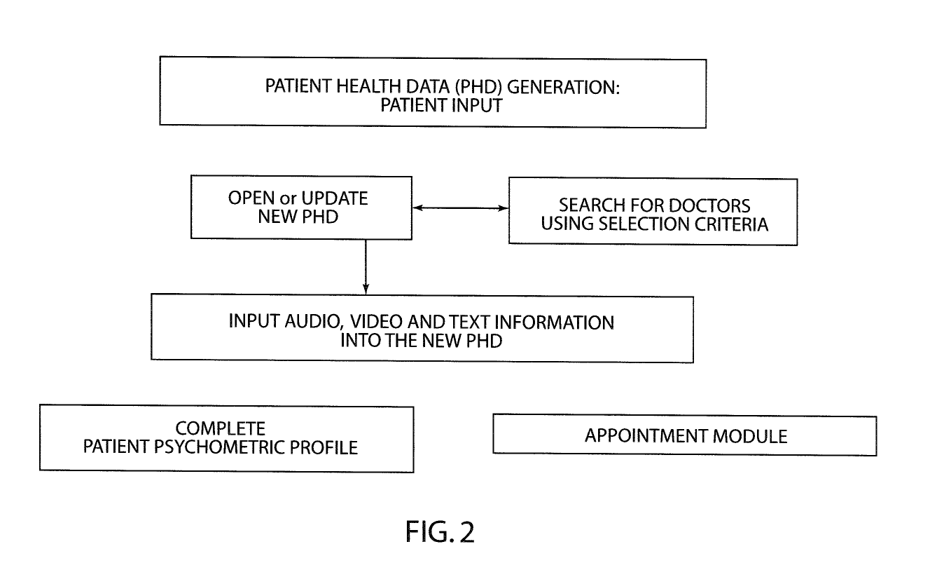

Clinical data entry will occur through many different routes; patient entry of information into the module can occur through voice dictation to a mobile device or pc, loaded digitally to a cloud server. Optionally the dictation or other message may then be transcribed by human transcriptionists or through an automated, digital transcription process and placed in the appropriate PHD. Typically non-quantifiable data such as subjective impressions of the patient's condition can be entered through voice dictation. FIG. 2 shows a schematic for patient input. The patient can search online for physicians using any one or more of a series of selection criteria. These selection criteria can comprise, for example, at least one of: location of the HCP; distance of the HCP from the patient user; distance of the HCP from a landmark; distance of the HCP from specific merchants or categories of merchants; distance of the HCP from transportation landmarks such as bus stops or train stops; insurance options accepted by the HCP and healthcare plans provided by the HCP; a medical specialty and/or subspecialty of the HCP; HCP availability indicator dependent on specific time slots searched; and doctor experience level. Other selection criteria will be readily apparent to those skilled in the art given the benefit of this disclosure. Of course the HCP profiles may be searchable via an internet search which will help the patient locate a specific, desired health care provider.

Separately, the patient may create a new patient health data module or update an existing one. Audio, video and text information may be input into the PHD. Common information included in the PHD would be, for example, a list of medications used by the patient, a history of past procedures of the patient, a physical condition of the patient, the patient's race, age, sex and religious affiliation. All of this information may be included in the PHD by the patient, or by the HCP, or both in combination and at different times. It is envisioned that commonly a patient would record an audio message for the HCP, often describing symptoms. Such patient-generated non-quantifiable data may advantageously be transcribed and entered into the patient health data. Transcription may occur by a human transcriptionist or by a computer-assisted transcription via voice transcription software. Optionally the amount of time of an audio message for a patient generated audio message may be restricted (for example, to 30 seconds) so as to limit costs of transcription and focus the patient on succinctly describing his symptoms. As noted in FIG. 2, the patient may also use an appointment module on the system which allows the patient to schedule a time to see the doctor. Once selected, an HCP can have access to the patient's medical information if so selected by the patient. In addition, the patient can select to have multiple HCPs consult on his or case. The system also allows the patient to select the primary HCP for a given case, and also to remove HCPs from his case if they no longer want a given HCP to be a consultant on their case. Further, as described in greater detail below, the patient may complete a psychometric profile which can be compared and scored against physician psychometric profiles to help the patient select a physician that he would be more comfortable with.

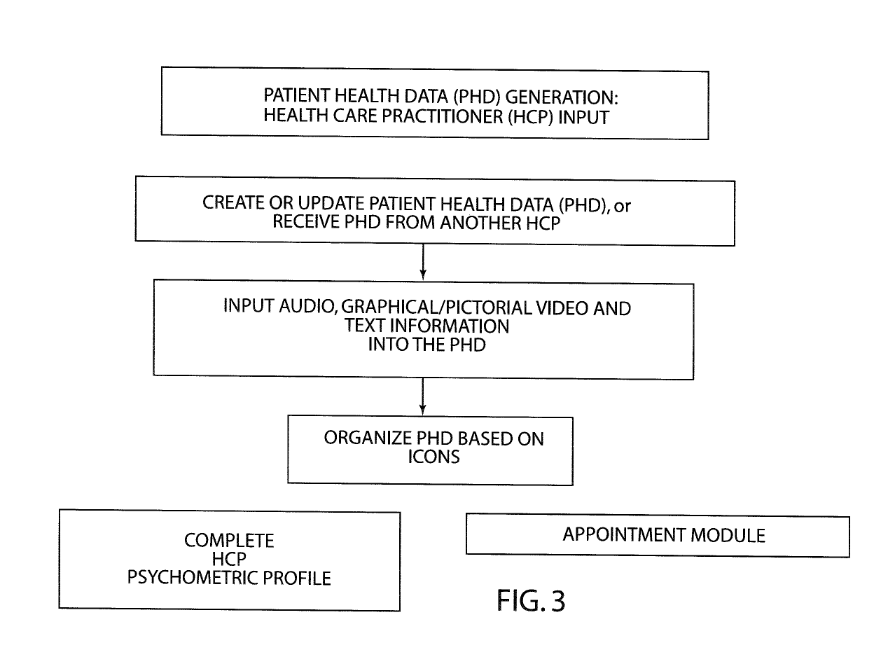

FIG. 3 is a schematic flow chart of health care practitioner (HCP) entry of data into the patient health data. First, either an existing PHD is accessed (with patient permission, especially when the patient is a new patient), or a new PHD is created with information provided from the patient. The PHD record advantageously can be readily transferred to another doctor, as in the case where a general practitioner refers a patient to a specialist, for example. Both non-quantitative and quantitative data can be added to the PHD by the health care practitioner.

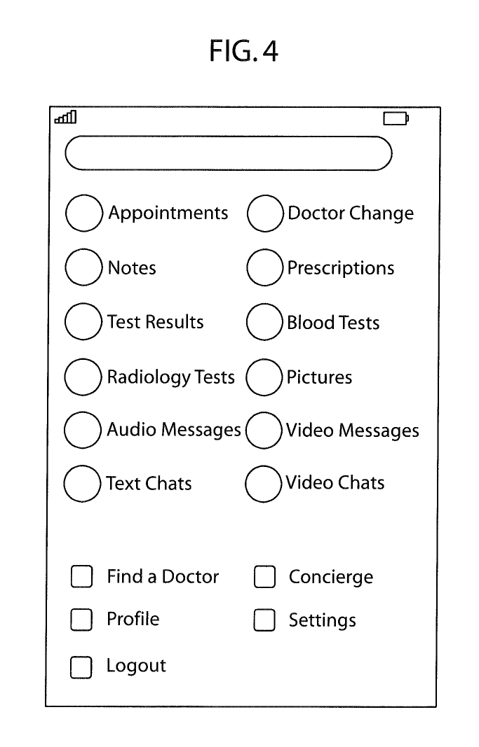

Non-quantitative data such as audio messages can be recorded via voice dictation, in a manner familiar to many doctors. Quantitative data such as test results can be uploaded and made accessible to the physician under, for example, a test results icon on the physician's mobile device as shown in FIG. 4 and discussed in greater detail below. Advantageously, the voice dictation of the HCP may be transcribed remotely and transferred to the PHD on the server. Generally transcription would be available for the physician, but may be merely one of several options for patients.

In accordance with another highly advantageous feature, when the quantitative data is graphical patient health data, such data may be received by the module and used to create a graph of the graphical patient health data for display on the corresponding computer of the HCP and/or of the patient. The system may be set to search by keyword (or keyphrase), effectively sensitizing the system to tag certain keywords in non-quantifiable data. Such keywords can be entered alongside quantifiable health data that would be suitable for graphical representation. Once the system recognizes a keyword, it searches for quantifiable data around such keyword and then generates a graph. The graph can be generated by linking up with other such instances of the same type of data. For example, regular measurements of blood pressure may be represented graphically, showing changes over time.

The appointment module of FIG. 2 allows patients to schedule appointments with the HCP via their (internet-connected) computer. For the health care providers, the portion of the appointment module noted in FIG. 3 may show additional information, and allow for and indicate cancellations and rescheduling.

Data entry for patients may be arranged so as to be entered and viewed chronologically, in the manner of a health log. For professionals, the patient health data may be entered into and viewed on a laptop or mobile device in a dashboard-like construct. In accordance with a highly advantageous feature, FIG. 4 shows a representative embodiment of a display of patient health data. Advantageously, the PHD may be presented to the health care practitioner segregated into sections by a series of icons. The section sections are accessed and are searchable by icons presented on the corresponding computer of the HCP. The icons presented can comprise at least one of appointments, notes, test results, radiology tests, audio messages, text chats, doctor change, prescriptions, blood tests, video messages, video chats, find a doctor data, profile, logout, concierge and settings. Also, an icon may be provided for patient billing, showing a record of charges and payments. Other icons suitable for use on the mobile clinical platform described herein will be readily apparent to those skilled in the art, given the benefit of this disclosure.

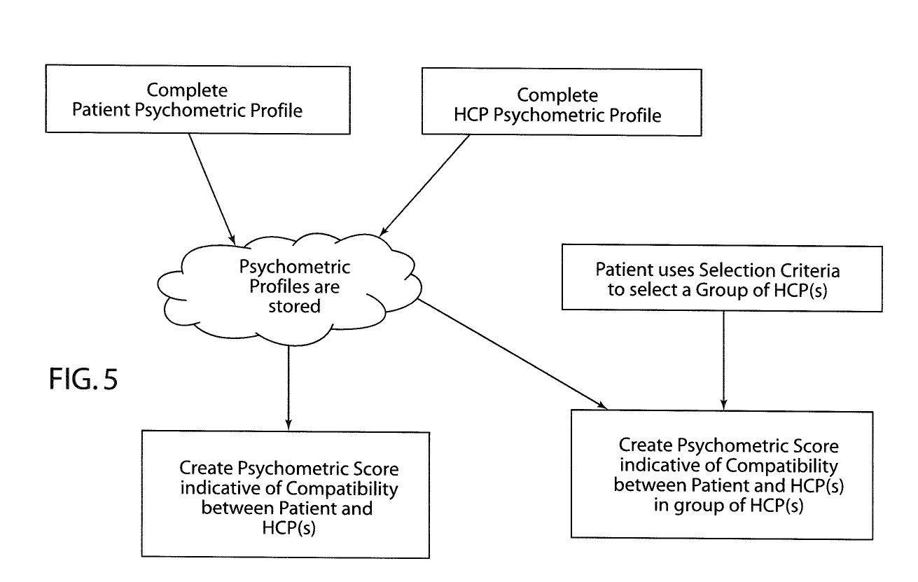

Even though a particular doctor is highly competent, some of his patients may nevertheless find him disagreeable to work with. This has the potential to reduce effective treatment of a patient. That is, a patient may be more likely to report symptoms accurately, and to follow a prescribed regimen of treatment if the patient likes the physician. Therefore, it is desirable to match patients and physicians based on characteristics that have been shown to increase satisfaction in interpersonal relationships. FIG. 5 shows another highly advantageous module for enhancing the doctor-patient relationship. A psychometric score indicative of compatibility between the patient and the health care provider is created based on a comparison of the patient psychometric profile and the health care provider psychometric profile previously created. Such profiles may be stored on the server as part of the PHD. Alternatively, the HCP profile may be stored separately and accessed as needed for a given patient. Information about the psychometric score may be presented to the patient to help him make his selection of a physician. These psychometric profiles serve as pre-screening agents for patients of healthcare practitioners, especially doctors (whose interaction with patients typically is very important).

The module presents to the patient a list of healthcare practitioners ranked based on the calculated estimate that the patient will have a satisfactory interaction with the given healthcare practitioner. The estimate is generated through preparation of psychometric profiles prepared by the health care practitioners and by the patients, both of which can be stored with the rest of the patient health data on a cloud server and accessed remotely. In operation, a new user of this service completes his patient psychometric profile, and the health care provider also completes his health care provider psychometric profile. Relevant characteristics of each profile can be used to generate a psychometric matching score indicative of compatibility.

The system then generates scores for relevant characteristics within the profile. Once this profile is generated, it is stored with the PHD on the database/server. When the user accesses the system to search for a healthcare provider, the system will match the user with the provider most likely to generate a satisfactory interaction based on the psychometric profiling. This match will occur either after a primary narrowing process has occurred (user selects specialty, location, etc.) or independent of such a primary step. The system can function via a computer algorithm or a human interviewer/assessor. For example, a computer algorithm based on Item response theory (IRT) is a psychometric approach which assumes that the probability of a certain response is a direct function of an underlying trait or traits. So a series of questions can be tailored to reflect personality traits. Once a matching algorithm is selected, the system uses that algorithm to rank the plurality of health care providers' profiles into a ranked list based on a comparison with the psychometric profile of the patient (as well as with any selection criteria). Finally, the system sends the ranked list to the patient.

The aforementioned selection criteria of the patient, used by the patient to limit the number of choices of physician (as by area, for example), may be used in combination with the psychometric profiles to further limit the selection to a group of health care providers with profiles which can be compared with the patient profile. A ranking of psychometric scores of the plurality of health care providers can be provided to the patient. In any case, the psychometric score is based on characteristics of the patient and of the health care provider, and the characteristics can comprise, for example, at least one of a level of a caring disposition (a bias toward taking time out to explain, give instructions or model behavior), a level of positivity (a bias toward thinking every desired outcome is achievable), a level of relationship perception (a bias toward paying close attention to information gathered through interactions and value knowing what is important to the other party), a level of leadership drive (a bias toward wanting to step forward and take control of a situation), and a level of inducement (a bias toward wanting to illustrate a point of view and getting others to want to accept their point of view). Also, in response to changes in doctor profiles, a new ranked and filtered list may automatically be generated by the module.

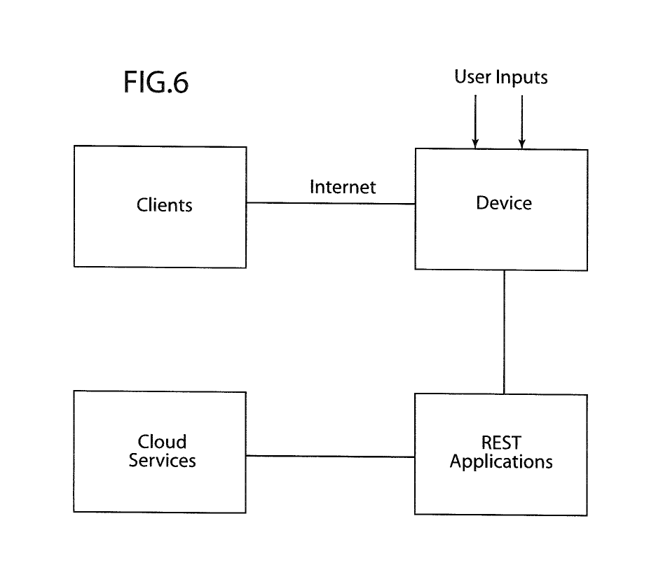

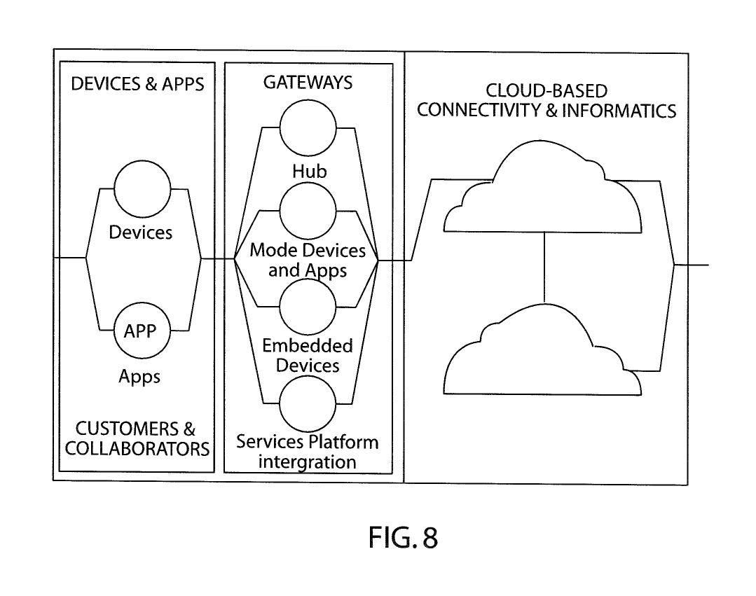

FIG. 6 is a schematic back end view of the module of FIG. 1, based on a platform of a cloud server (FIG. 8) and mobile devices. In accordance with this embodiment, the patient health data may be entered and accessed via a smartphone, tablet, iPad, laptop, etc. Generally a relatively lightweight computer with (typically wireless) access to the internet is preferred. Thus, the module takes advantage of cloud computing. Cloud computing is the use of computing resources (hardware and software) that are delivered as a service over a network (typically the Internet). The name comes from the common use of a cloud-shaped symbol as an abstraction for the complex infrastructure it contains in system diagrams. Cloud computing entrusts remote services with a user's data, software and computation. End users (here, patients and health care providers) access the module containing the patient health data through a web browser or a light-weight desktop or mobile app while the patient health data and related software are stored on servers at a remote location. FIG. 8, shows a cloud module. A cloud computing module advantageously allows hospitals and physicians to avoid upfront infrastructure costs, and focus on patient treatment.

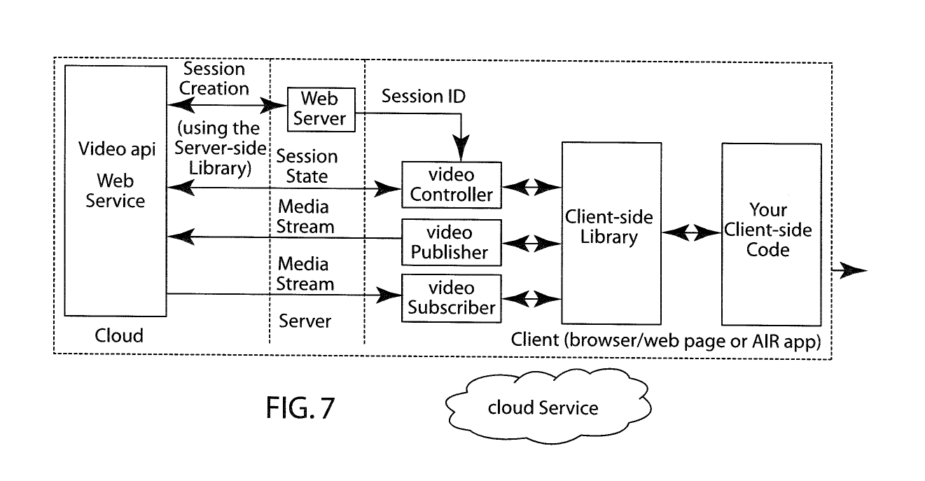

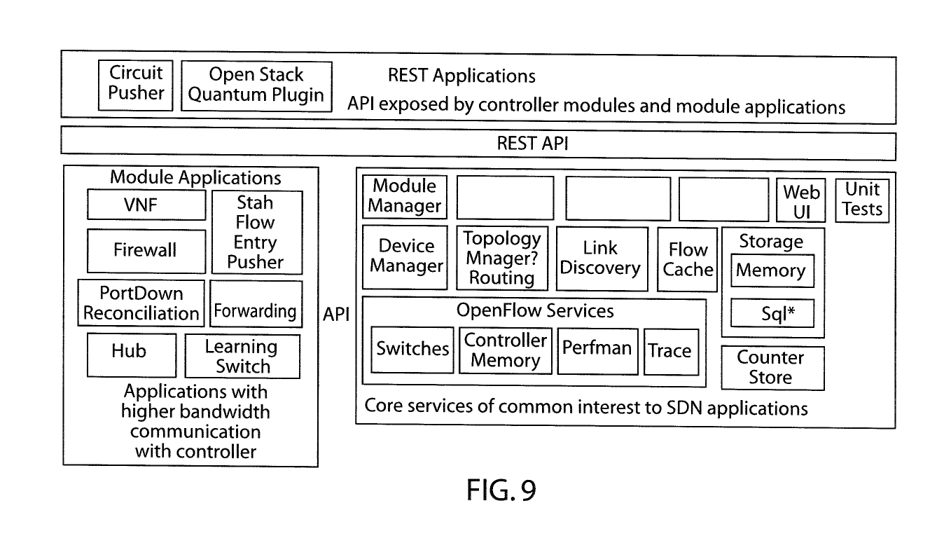

The devices used preferably are designed to be compliant with the Health Insurance Portability and Accountability Act of 1996 (HIPAA) and interoperable with Health Level Seven (HL7) compatible systems. HL7 is a non-profit organization involved in the development of international healthcare informatics interoperability standards. HL7 also refers to some of the specific standards created by the organization (e.g., HL7 v2.x, v3.0, HL7 RIM). The module is preferably a cloud based client server system. A uniform interface separates clients from servers. The client module is shown separated from the servers in FIG. 6, and is shown in more detail in FIG. 7. This separation of concerns means that clients are not concerned with data storage, which remains internal to each server, so that the portability of client code is improved. Servers are not concerned with the user interface or user state, so that servers can be simpler and more scalable. Servers and clients may also be replaced and developed independently, as long as the interface between them is not altered. FIG. 6 also shows REST Application programming interfaces. REST refers to Representational State Transfer, and is a predominant style of software architecture for distributed systems such as the World Wide Web. The platform for presenting healthcare information disclosed herein can also have communication modules (SMS, Voice, etc.). Preferably the platform is made compliant with consortia like the Continua Health Alliance, the industry organization of healthcare and technology companies that collaborate to improve the quality of personal healthcare. Such compliance, in combination with communication modules enables other medical devices to be readily integrated and connected to the platform. See FIG. 9 for a more detailed description of a representative example of some of the elements of REST API used in this embodiment.

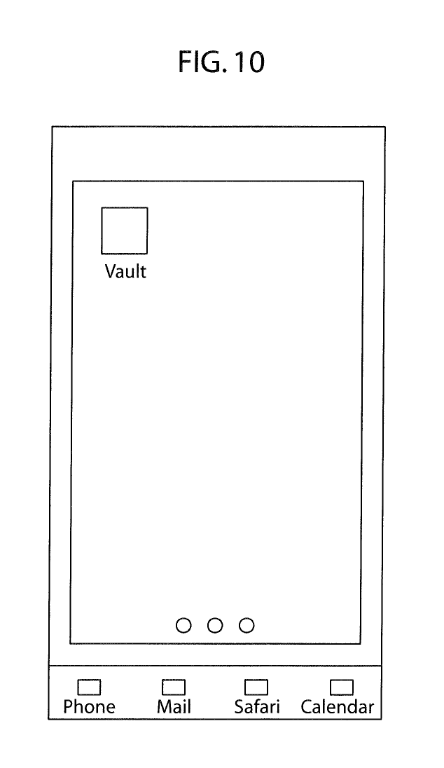

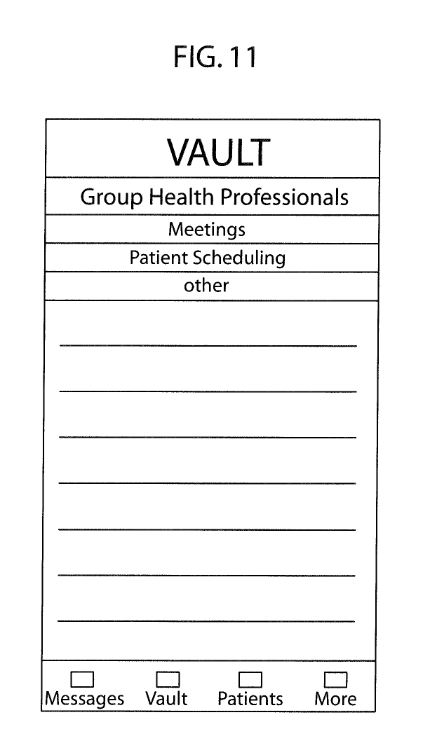

FIG. 10 is a schematic example of a button or app on a mobile device for running another advantageous element of the module, an administrative module. The administrative module may be accessible by pressing on the screen of a mobile device, pc or laptop at the icon in a conventional manner. In the embodiment of FIG. 10, this module is labeled Vault, although other names can be used for administrative data. The administrative module may comprise messages and information and other data created by and administrator, in text, pictorial and/or video format, which may be delivered to a single health care provider, a single patient, or to a plurality of health care providers and/or patients. In the example shown in FIG. 11, the administrative module is used in a group practice, and a plurality of physicians in the group may each be notified of an upcoming event, such as a meeting, a continuing medical education seminar, or a group practice meeting. The physicians may also be individually notified of events, such as, for example, their patient schedule. Other items may be delivered to the physicians in the group practice. For ease of design, the administrative information presented may be “pushed” out to the health care providers (or to the patients, or both) in the sense that such information is delivered essentially one way, and no return data is sent back. Alternatively, administrative information may be delivered back to the administrator, as for example, an indication that a particular person will attend a scheduled meeting.

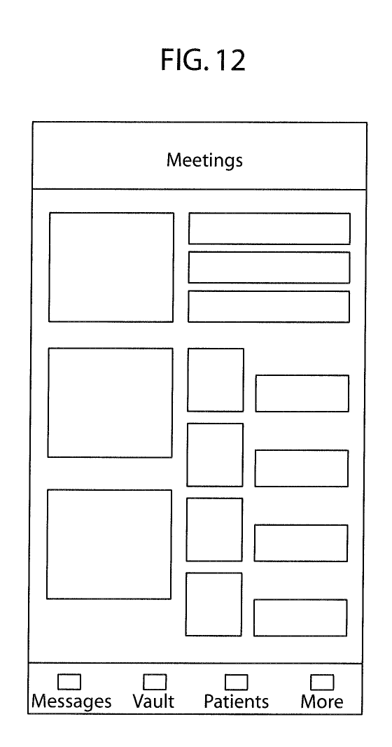

FIG. 12 shows a simplified schematic of a representative image present on a mobile device when the meeting tab shown in FIG. 11 is selected. More detailed information about the meeting may be shown, such as a title of the meeting, location, date, speaker, information about the speaker, registration information etc.

The platform for presenting health care information disclosed herein advantageously not only allows for ease of use, but also has the potential to improve patient outcomes. For example, a patient may be seen having a condition with ambiguous symptoms, and a doctor may prescribe a test. The test results may clearly show the cause of the symptoms, and the sooner the test results are in the hands of the physician, the sooner treatment can begin. Where the condition is life threatening, rapidly presenting the test results can potentially save the life of the patient.

From the foregoing disclosure and detailed description of certain embodiments, it will be apparent that various modifications, additions and other alternative embodiments are possible without departing from the true scope of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of presenting health care information, comprising, in combination, the steps of:
creating patient health data for a patient, and storing the patient health data on a server;
accessing the patient health data by one of the patient and a health care provider on a corresponding computer, wherein the corresponding computer is connected to the server over the internet;
presenting the patient health data accessed in sections searchable by icons presented on the corresponding computer;
wherein the patient health data comprises non-quantifiable data from one of either and both the patient and the health care provider; and
the icons presented comprise at least one of appointments, notes, test results, radiology tests, audio messages, text chats, doctor change, prescriptions, blood tests, video messages, video chats, find a doctor, profile, logout, concierge and settings.

2. The method of claim 1 wherein the health care provider-generated non-quantifiable data is an audio message, and further comprising the step of transcribing the audio message to enter the audio message into the patient health data.

3. The method of claim 1 wherein the patient-generated non-quantifiable data is an audio message, and further comprising the step of transcribing the audio message to enter the audio message into the patient health data.

4. The method of claim 3 further comprising the step of restricting the amount of time of an audio message for a patient generated audio message.

5. The method of claim 3 wherein the step of transcribing comprises auto-transcription of the audio message.

6. The method of claim 1 further comprising the steps of receiving graphical patient health data and creating a graph of the graphical patient health data for display on the corresponding computer.

7. The method of claim 6 further comprising the step of searching by keyword in the non-quantifiable data to obtain the graphical patient health data.

8. The method of claim 1 wherein the patient health data comprises at least one of audio messages, live chat, audio-video streaming systems, electronic medical records, hospital information system data, medical images, lab information, scheduling data, and psychometric profiles.

9. The method of claim 8 wherein the audio message is voice dictation, and further comprising the step of transcribing the voice dictation from the health care provider.

10. The method of claim 1 wherein the corresponding computer of the health care provider is adapted to be wirelessly connected to the Internet.

11. The method of claim 1 further comprising the step of creating a health care provider profile by a health care provider which comprises information about the health care provider, including contact information which is searchable by the patient.

12. The method of claim 1 further comprising the step of restricting access by a health care professional to patient health data for a given patient until permission from the given patient is received.

13. The method of claim 1 further comprising the step of creating administrative data and delivering the administrative data to one or more of the patients and the health care providers.

14. The method of claim 13 wherein the administrative data comprises a patient schedule and information about an upcoming meeting.

15. The method of claim 1 further comprising the steps of:
creating patient health data for a patient on a processor of a patient computer, and storing the patient health data on a processor of a server remote from the computer, wherein the patient health data comprises a patient psychometric profile;
creating a health care provider psychometric profile on a health care provider computer which can be accessed remotely by the server holding the patient health data;
creating a psychometric score indicative of compatibility between the patient and the health care provider based on a comparison of the patient profile and the health care provider profile, wherein both the patient psychometric profile and the health care provider psychometric profile measure a personality trait of the individual completing the profile, each patient creates one of the patient psychometric profiles, the health care provider creates the health care provider psychometric profile, and the psychometric profiles are based upon Item response theory such that the comparison increases satisfaction in interpersonal relationship between the patient and the health care provider; and
presenting information about the psychometric score to the patient.

16. The method of claim 15, further comprising the step of using selection criteria selected by the patient to limit a group of health care providers with profiles which can be compared with the patient profile;
wherein the selection criteria comprise at least one of
a location of the health care provider;
a distance of the health care provider from one of the patient, a landmark, a specific merchant or a category of merchants;
an insurance option provided by the health care provider;
a medical specialty of the health care provider;
an availability of the health care provider to meet with the patient; and
a doctor experience level.

17. The method of claim 15 wherein the psychometric score is based on characteristics of the patient and of the health care provider, and the characteristics comprise at least one of a level of a caring disposition, a level of positivity, a level of relationship perception, a level of leadership drive, and a level of inducement.

18. The method of claim 15 wherein the step of creating a psychometric score indicative of compatibility between the patient and the health care provider based on a comparison of the patient profile and the health care provider profile comprises creating a psychometric score between the patient and a plurality of health care providers; and
the step of presenting information about the psychometric score to the patient comprises presenting a ranking of psychometric scores of the plurality of health care providers.
